(19) United States
(12) Patent Application Publication
(10) Pub. No.: US 2025/0270267 A1
(43) Pub. Date: Aug. 28, 2025

(54) METHOD FOR PRODUCING AT LEAST ONE FIBROIN SOLUTION

(71) Applicant: Fibrothelium GMBH, Aachen (DE)

(72) Inventors: Alexander Kopp, Aachen (DE); Marius Köpf, Erkelenz (DE); Phillip Czichowski, Aachen (DE)

(21) Appl. No.: 18/705,004

(22) PCT Filed: Nov. 28, 2022
(86) PCT No.: PCT/EP2022/083427

§ 371 Date: Apr. 26, 2024

(30) Foreign Application Priority Data
Dec. 3, 2021 (DE) 10 2021 131 918.8

(51) Int. Cl. C07K 14/435

(52) U.S. Cl. CPC C07K 14/43586

(57) ABSTRACT

A method for generating at least one fibroin solution with the following method steps proceeding in the indicated order:
providing fibroin;
dissolving the fibroin in a first solution system to generate a fibroin raw solution;
cleaning the fibroin raw solution by lowering the pH of the fibroin raw solution by introducing an acidic substance into the fibroin raw solution;
raising the pH of the fibroin solution by introducing a basic compound into the fibroin raw solution;
desalting the fibroin raw solution to produce the fibroin solution.

The invention relates to a method for producing at least one fibroin solution, in particular from silk and in particular in a medical quality.

Silk is a natural product which, due to its unique profile of properties, is not only processed in the textile industry but also has large application potential for life sciences, in particular for medical technology. In particular fibroin which, as a protein, is a building block of natural silk has properties which predestined it for use as a medical product. However, the problem arises in this regard of providing fibroin in a usable form and for use as medical quality medical product.

Such a provision of fibroin is the object on which the invention is based.

This object is achieved by a method by which at least a fibroin solution is also produced, wherein the method comprises at least the following method steps proceeding in the indicated order:
providing fibroin; fibroin is understood in the context of the invention to be a structural protein which consists of amino acids (preferably glycine and/or serine and/or alanine) and also is of natural and/or synthetic origin; the provision can be, for example, in the form of cocoons, or wool, or fibers, or powder (natural origin or recombinantly produced);
dissolving the fibroin in a first solution system for generating a fibroin raw solution; dissolving in the context of the present invention means converting a certain substance, such as fibroin, into a flowable, preferably homogeneous, at least apparently homogeneous form. In the context of the present invention, a solution system denotes a combination of chemicals which can be used to dissolve a certain substance.
purifying the fibroin solution by lowering the pH of the fibroin solution, preferably to a value of <2, preferably <1.5, particularly preferably <1, by introducing at least one acidic substance; which here corresponds to a substance having a pH value of <4, preferably <2, particularly preferably <1 in the fibroin raw solution; the acidic substance can preferably be an acid, for example formic acid, and/or acetic acid, and/or citric acid, and/or hydrochloric acid; the acidic compound can be introduced in solution with a solvent, preferably water, particularly preferably distilled water having a conductivity of less than 30 μS/cm; when such a solution is used, the concentration of this solution can be, for example, between 0.1 M and 6 M, preferably approximately (i.e., ±0.1 M) or exactly 1 M or 0.5 M; the cleaning causes the deactivation and/or destruction of unwanted impurities and a reduction of contaminants such as, for example, fungal spores, endotoxins or bacteria; this has the advantage that the fibroin raw solution can also be processed without cleaning steps, such as for example sterilization, which are downstream from a method according to the invention, for example of medical devices and in particular medical implants;
raising the pH of the fibroin solution, in particular to a value between 6 and 9, preferably between 7 and 8 and particularly preferably to a value of approximately (i.e., ±0.1) or exactly 7.5, by introducing a basic substance (i.e., a substance having a pH of >10, preferably >11, particularly preferably >12), preferably sodium carbonate (Na₂CO₃), or sodium hydroxide (NaOH), or potassium hydroxide (KOH), or magnesium hydroxide (MgOH), or calcium hydroxide (CaOH) in the fibroin raw solution; the basic compound can be introduced in solution with a solvent, preferably water, preferably distilled water having a conductivity lower than 30 μS/cm; when such a solution is used, the concentration of this solution can preferably be between 0.1 M and 6 M, preferably approximately (i.e., ±0.1 M) or exactly 1 M or 0.5 M if possible;
desalting (i.e., reducing the salt content) of the fibroin raw solution to produce the fibroin solution.

“Fibroin” is also understood to mean a substance comprising (pure) fibroin.

The method can comprise further preceding and/or subsequent method steps and/or intermediate steps.

Providing the fibroin in the form of a fibroin solution permits easy and advantageous use, for example for the production of medical products, such as for example scaffolds, membranes, wound dressings, bandages, hydrogels, adhesives, powders, and fillers, or for coating medical devices. By a targeted variation of the process parameters within certain limits, the solution properties such as for example the viscosity or the molecular weight of the protein solution can also be precisely adapted to downstream further processing. In particular, a high-viscosity fibroin solution can also be produced dependent on the concentration, preferably with a viscosity between 300 mPa*s and 1500 mPa*s, preferably between 300 mPa*s and 600 mPa*s or between 1200 mPa*s and 1500 mPa*s, which can advantageously be used within the scope of further use of the fibroin solution for producing highly stable structures. The viscosity can be determined according to DIN 53019-1.

In the context of a method according to the invention, the cleaning and also the desalinization of the fibroin solution ensure sufficient purity of the fibroin solution, wherein a medical quality (“medical grade”) can in particular also be achieved. The term “medical quality” is understood to mean that the fibroin solution, at least at a concentration of up to 5 wt. %, contains a bioburden <1 CFU/ml and endotoxins with a content <0.5 EU/ml. At a concentration of greater than 5 wt. %, the bioburden can correspondingly increase proportionately (ratios according to a concentration of 5 wt. % on the one hand and, on the other hand, the bioburden is <1 CFU/ml and endotoxin is <0.5 EU/ml). In this context, “bioburden” denotes the number of colony-forming germs or bacteria in a volume unit of the solution. For this purpose, it may be necessary for at least the cleaning of the fibroin solution and the subsequent process steps to be carried out under sterile conditions. Sterile conditions are working conditions such as can be achieved when sterilized tools and accessories in an environment, which prevents contamination of the fibroin solution (e.g., sterile workbench). After the cleaning step, the fibroin solution should not be contaminated by contaminants, i.e., by an entry of inorganic, and/or organic, and/or biological impurities in the form of, for example, fungal spores, endotoxins or bacteria. The endotoxin content can be determined according to DIN EN ISO 29701. The bioburden can be determined according to DIN EN ISO 11737-1.

The dissolution of the fibroin can preferably be assisted by introducing thermal energy, for example by heating to a temperature (and maintaining the temperature) of 70° C., or 75° C., or 80° C., or 90° C. or 100° C., and/or by treatment with microwave radiation, for example for 15 minutes at a power of 300 W, or for 10 minutes at a power of 400 W, or for 5 minutes at a power of 500 W; and/or with infrared irradiation, and/or by introducing kinetic energy, for example by ultrasonic treatment or stirring or shaking, of the mixture comprising the fibroin and the first solution system, or the fibroin raw solution that forms.

The first solution system can preferably be an acidic compound, in particular formic acid (CH₂O₂) or acetic acid (CH₃COOH), or a chaotropic salt compound, in particular calcium chloride (CaCl₂)), or crystal water/calcium chloride (CaCl₂)-2H₂O), or lithium bromide (LiBr), or zinc chloride (ZnCl₂), or calcium nitrate (Ca₂NO₃), the latter preferably in the form of a melt which can be obtained, for example, after 30 minutes at 40° C. in a water bath. In addition, the first solution system can comprise a solvent, in particular methanol (CH₄O), and/or acetone (C)₃H₆O), and/or ethylene glycol (C)₂H₆O₂) and/or ethanol (EtOH), which improves the dissolving process by means of a swelling effect. Furthermore, the first solution system can comprise water, preferably distilled water, preferably ultrapure water without (biological) contamination. For example, it can be provided that the first solution system is a chaotropic salt compound, a solvent and optionally distilled water in a defined ratio, for example in a molar ratio of calcium chloride:ethanol:water of 1:2:8, or of crystal water/calcium chloride:ethanol:water of 1:2:6 or 1:4:6, or of calcium nitrate:ethanol of 1:4, or 1:3, or 1:2. Especially when calcium chloride and crystal water/calcium chloride are used as a constituent of the first solution system, a fibroin solution can be realized which can be processed to form water-insoluble structures. In the present context, water solubility denotes the degree of dissolution and thus the property of a solid body structure in contact with water in a flowable and preferably also homogeneous form. Structures are defined herein as water-soluble, which dissolve in water to more than 80% of their original mass. Structures that dissolve in water between 30% and 70% of their original mass are limitedly soluble in water. Structures which dissolve in water to less than 30% of their original mass are defined herein as water-insoluble.

The cleaning of the fibroin raw solution can preferably be carried out over a period of at least 12 h and/or at most 72 h, for example over a period of approximately (i.e., ±1 h) or exactly 24 h, or 20 h, or 16 h.

The desalting of the fibroin raw solution can be brought about by means of filtration and/or preferably by means of dialysis, wherein during dialysis, salt molecules diffuse through a semi-permeable membrane from the fibroin raw solution into a recipient liquid, preferably into purified desalted water, which can be acidic, or neutral or basic, and/or into polyethylene glycol (PEG) with a molecular size of more than 10 kDa and are thereby removed from the fibroin raw solution. By using polyethylene glycol (PEG), the additional advantage can be realized here that, by generating an osmotic pressure, a direct concentration of the protein solution takes place from water exiting. Desalted water is understood to mean water with a conductance below 50 μS/cm, preferably below 30 μS/cm.

Desalting by means of dialysis can be accelerated by generating a volume flow of the fibroin raw solution, for example in combination with a pump performance of 50 ml/min to 500 ml/min, preferably 200 ml/min, or 300 ml/min, or 400 ml/min, in the form of diafiltration.

The desalting of the fibroin raw solution can preferably be carried out until a conductivity value of the fibroin solution is reached of a maximum of 1000 μS/cm or a maximum of 750 μS/cm, or preferably a maximum of 500 μS/cm, or particularly preferably a maximum of 250 μS/cm.

A concentration of the fibroin in the fibroin solution can preferably be set by generating an overpressure (relative to ambient pressure) of the fibroin raw solution during the desalting, preferably an overpressure of at most 5 bar, preferably of approximately (i.e., ±0.1° bar) or (as far as possible) exactly 3 bar, or 2 bar or 1 bar. By a corresponding adjustment of the overpressure of the fibroin raw solution, so-called diafiltration can hence be achieved so that the fibroin raw solution (and hence the resulting fibroin solution) is not only desalted but also concentrated. This can preferably be carried out up to a concentration of the fibroin of at least 3% or 10% or, preferably, 20% or, particularly preferably, 30%. The overpressure can be produced, for example, by reducing a flow cross section by which the fibroin raw solution is conveyed, or by increasing the conveyed mass flow of the fibroin raw solution.

The viscosity of the fibroin solution to be produced can be influenced by a targeted adjustment of the pH of the fibroin raw solution to which it was raised after cleaning. A pH<5 can thereby result in a viscous solution which transitions into a solid gel even after brief storage. At a pH between 5 and 8, the resulting solution can be very viscous, but storable and processable. As of a pH of 8, a solution arises which, despite high concentrations of the fibroin of, for example, up to 30 wt. %, has a relatively low viscosity behavior.

According to a preferred embodiment of a method according to the invention, it can be provided that the provision of the fibroin is brought about by separating the fibroin and at least one secondary constituent of a silk. In the context of the invention, the term silk is understood to mean a mixture of substances, wherein fibroin makes up at least one substantial constituent (i.e., at least 30 wt. %) and preferably the main component (i.e., at least 50 wt. %). The separating can be brought about by introducing the silk, for example in the form of immersing one or more silk cocoons, into a second solution system which selectively dissolves the secondary constituent, as a result of which a mixture (i.e., a heterogeneous mixture) consisting of the (solid) fibroin and the solution with the secondary constituent arises, and by removing the fibroin from the second solution system, for example by filtering by means of a filter or sieve.

Such a process is also referred to below as “degumming”. This represents easy to perform option for providing fibroin from silk that is a natural supplier for fibroin. This makes it possible in principle to use any silk, in particular any silk of a silk-producing creature, preferably the silk of the silkworm Bombyx Mori, as a starting product for carrying out a method according to the invention.

Particularly preferably, it can be provided that the secondary constituent is sericin which, in addition to the fibroin, is contained for example in the silk, which is produced by the silkworm Bombyx Mori.

The second solution system preferably used for separating the fibroin and the at least one secondary constituent can preferably be a basic substance, for example sodium carbonate (Na₂CO₃), or sodium hydroxide (NaOH), or potassium hydroxide (KOH), or magnesium hydroxide (MgOH), or calcium hydroxide (CaOH), or urea (CH₄N₂O). The concentration of the basic substance can preferably be between 0.01 M and 8 M, preferably approximately (i.e., ±0.01 M) or (as far as possible) exactly 1 M or 0.5 M.

When sodium carbonate is used, it can preferably be provided that the mixture comprises the sodium carbonate, the fibroin and distilled water in a ratio which approximately (i.e., ±5% per value), or (as far as possible) exactly, corresponds to a ratio of either 0.06 M or 0.04 M or 0.02 M sodium carbonate to 0.2 kg fibroin to 6 l distilled water.

The separation of the fibroin and the at least one secondary constituent can preferably be supported by introducing thermal energy into the mixture, preferably until the temperature of the mixture of between 70° C. and 100° C., in particular of approximately (i.e., ±2° C.) or (as far as possible) exactly 70° C. or, 80° C., or 90° C., or 100° C. is reached (and held). For this purpose, it can be provided, for example, that the mixture is heated over a period of either at least 20 minutes (preferably between 25 minutes and 35 minutes, in particular 30 minutes) or from at least 50 minutes (preferably between 55 minutes and 65 minutes, in particular 60 minutes) to a temperature of at least 90° C. (preferably at least 95° C., particularly preferably 100° C.). If the second solvent comprises sodium hydroxide, in particular in the form of sodium hydroxide solution, the introduction of thermal energy can also be dispensed with, which can simplify a method according to the invention. When urea is used, the heating time could also be up to three hours.

Alternatively or additionally, the separation of the fibroin and the at least one secondary constituent can be assisted by introducing kinetic energy into the mixture, preferably by ultrasound treatment or by stirring or shaking (generating vibrations) or by swinging.

The fibroin can preferably be washed and/or dried after removal from the solution with the secondary constituent, in particular the sericin solution, and before dissolving in the first solution system. The washing can preferably be accomplished by rinsing with desalted or pure water. Desalted water in this case is understood to be water having a conductance below 50 μS/cm, preferably below 30 μS/cm; pure water is understood to be water which is suitable for intravenous medical applications. The drying can be carried out, for example, in an oven, preferably at a temperature of at least 70° C. and/or at most 100° C., preferably at a temperature of approximately (i.e., ±2° C.) or (as far as possible) exactly 80° C. Rapid drying, for example within 5 hours, can thereby be realized. Drying at temperatures of at least 70° C. also promotes deactivation and/or destruction of biological impurities.

According to a preferred embodiment of a method according to the invention, it can be provided that one, more or all of the following method steps are carried out:
purifying the solution with the secondary component by lowering the pH of the solution with the secondary component by introducing an acidic compound into the solution with the secondary constituent, and/or by heating the solution with the secondary component;
raising the pH of the solution with the secondary component by introducing a basic substance into the solution with the secondary component;
desalinating the solution with the secondary constituent up to a conductivity of at most 5000 μS/cm, or 3000 μS/cm, or 1000 μS/cm, or 500 μS/cm.

As a result, the secondary constituent, in particular sericin, an adhesive protein found in the silk of the silkworm Bombyx mori, which holds the fibroin together and makes up 20 wt. % to 30 wt. % of the silk, can also be used advantageously, in particular also with a medical quality and therefore as a medical product or for cosmetics.

The process parameters and agents/products provided for purifying the solution with the secondary constituent by lowering the pH and for raising the pH of the solution with the secondary constituent and for desalinating the solution with the secondary constituent can be the same as those described for purifying the fibroin raw solution, and for raising the pH of the fibroin raw solution, and for desalinating the fibroin raw solution.

To the extent that “comprise” is mentioned in this description, this also thereby means to the possibility of an exhaustive list (i.e., “consists of”).

The invention is explained in more detail below with reference to exemplary embodiments.

### EXEMPLARY EMBODIMENT 1 (STANDARD FIBROIN SOLUTION 1)

For providing the fibroin, 200 g of silk cocoons of the silkworm Bombyx mori is boiled in a (second) solution system consisting of 6 l boiling water and 26.8 g sodium carbonate (0.04 M) for 60 minutes with regular stirring. As a result, the sericin of the silk cocoons was dissolved in the second solution system and thereby separated from the fibrous fibroin. The fibroin fibers were then removed from the sericin solution by a sieve.

This was followed by washing and drying the fibroin fibers by swelling them three times for 10 minutes with distilled water and then drying them at 80° C. in an oven for 4 hours (commercially available dehydrator).

The dried fibroin fibers were then weighed and distributed in equal parts to containers. A (first) solution system was then added to the container, wherein this solution system has ten times the mass relative to the fibroin fibers (30 g of fibroin fibers+300 g of first solution system). The first dissolution system used was a so-called Ajisawa solution, which consists of calcium chloride, ethanol and distilled water in a fixed ratio of 1:2:8 (see Ajisawa A. Dissolution of silk fibroin with calcium chloride/ethanol aqueous solution. J. Sericultural Sci. Jpn. 1998; 67:91-94.). By heating the mixture of fibroin fibers and solution system in a water bath at 75° C. and over a period of 60 minutes, the fibroin fibers were dissolved in the first solution system. The containers were shaken vigorously after 15 minutes in each case. In this way, a fibroin raw solution was produced.

For subsequent purification, the fibroin raw solution was treated for 24 h with 1 M HCl solution in an amount which was 1/10 of the mass of the dissolved fibroin. The pH of the fibroin raw solution was thereby lowered to a value of <2.

The subsequent neutralization by raising the pH of the fibroin solution to 7.5 was carried out under sterile conditions with 1 M NaOH solution, in the same amount as the HCl solution ( 1/10 of the mass of the dissolved fibroin), which was previously added to the fibroin raw solution.

For desalting, the purified fibroin raw solution was desalted in a sterile dialyzer (polyether sulfone (PES) membrane or cellulose membrane with a molecular cutoff of 5 to 10 kDa) until the waste water has a permanent conductivity of <500 μS/cm. The solution was concentrated to 30% (mass fraction of the fibroin in the resulting fibroin solution).

The solution resulting therefrom had the following typical properties:

| Appearance | Amber clear solution | |
|---|---|---|
| Molecular weight | 10-400 | kDa |
| pH | 7.75 | |
| Conductivity | 300 | μS/cm |
| Viscosity | 350 | mPa · s |
| Endotoxin value | <1 | EU/ml |
| Bioburden | <5 | CFU |
| DNA content (260:280 nm) | <0.65 | |
| Impurities (e.g., heavy metals) | <50 | ppm |

### EXEMPLARY EMBODIMENT 2 (STANDARD FIBROIN SOLUTION 2)

For providing the fibroin, 200 g of silk cocoons of the silkworm Bombyx mori is boiled in a (second) solution system consisting of 6 l boiling water and 26.8 g sodium carbonate (0.04 M) for 60 minutes with regular stirring. As a result, the sericin of the silk cocoons was dissolved in the second solution system and thereby separated from the fibrous fibroin. The fibroin fibers were then removed from the sericin solution by a sieve.

The fibroin fibers were then washed and dried by swelling them three times for 10 minutes with distilled water and then drying them in an oven at 80° C. for 4 h.

The dried fibroin fibers were then weighed and distributed in equal parts to containers. A (first) solution system was then added to the container, wherein this solution system has ten times the mass relative to the fibroin fibers (30 g of fibroin fibers+300 g of first solution system). The first solution system used was a so-called Ajisawa solution which consists of calcium chloride, ethanol and distilled water in a fixed ratio of 1:2:8. By heating the mixture of fibroin fibers and first solution system in a water bath at 75° C. and over a period of 60 minutes, the fibroin fibers were dissolved in the first solution system. The containers were shaken vigorously after 15 minutes in each case. In this way, a fibroin raw solution was produced.

For a first desalting, the fibroin raw solution was desalted by means of a dialyzer until the waste water of the dialyzer had a permanent conductivity of <500 μS/cm.

For subsequent purification, the fibroin raw solution was treated for 24 h with 1 M HCl solution in an amount which was 1/10 of the mass of the dissolved fibroin. The pH of the fibroin raw solution was thereby lowered to a value of <2.

The subsequent neutralization by raising the pH of the fibroin solution to 7.5 was carried out under sterile conditions with 1 M NaOH solution, in the same amount as the HCl solution ( 1/10 of the mass of the dissolved fibroin), which was previously added to the fibroin raw solution.

For a second desalting, the purified fibroin raw solution was desalted in the sterile dialyzer until the waste water had a permanent conductivity of <500 μS/cm. The solution was concentrated to 10% (mass fraction of the fibroin in the resulting fibroin solution).

The solution resulting therefrom had the following typical properties:

| Appearance | White, cloudy solution | |
|---|---|---|
| Molecular weight | 10-400 | kDa |
| pH | 7.75 | |
| Conductivity | 150 | μS/cm |
| Viscosity | 50 | mPa · s |
| Endotoxin value | <1 | EU/ml |
| Bioburden | <5 | CFU |
| DNA content (260:280 nm) | <0.65 | |
| Impurities (e.g., heavy metals) | <50 | ppm |

### Supplement to Exemplary Embodiment 2 (Sericin Solution)

The sericin solution remaining after removal of the fibroin fibers was desalted by means of the dialyzer until the waste water has a permanent conductivity of <500 μS/cm.

The sericin solution was then concentrated until the minimum volume of the dialyzer (200 ml) was reached.

The concentrated sericin solution was then purified, e.g., by autoclaving in a heat-stable container, or by microwave irradiation at 300 W for 10 minutes (allowed to cool briefly for every 3 minutes) or in accordance with the method according to the invention with 1 M hydrochloric acid.

The purified sericin solution was sterilely decanted after cooling.

The sericin solution resulting therefrom has the following typical properties:

| Appearance | Brownish, cloudy solution | |
|---|---|---|
| Molecular weight | 5-400 | kDa |
| pH | 8.3 | |
| Conductivity | 3000 | μS/cm |
| Viscosity | 15 | mPa · s |
| Endotoxin value | <5 | EU/ml |
| Bioburden | <10 | CFU |
| DNA content (260:280 nm) | <0.65 | |
| Impurities (e.g., heavy metals) | <100 | ppm |

### Embodiment 3 (Gel Fibroin Solution)

For degumming, 200 g of silk cocoons of the silkworm Bombyx mori is boiled in a (second) solution system consisting of 6 l boiling water and 13.4 g sodium carbonate (0.02 M) for 30 minutes with regular stirring. As a result, the sericin of the silk cocoons was dissolved in the second solution system and thereby separated from the fibrous fibroin. The fibroin fibers were then removed from the sericin solution by filtration by means of a sieve.

This was followed by washing and drying the fibroin fibers by swelling them three times for 10 minutes with distilled water and then drying them at 80° C. in an oven for 4 hours (commercially available dehydrator).

The dried fibroin fibers were then weighed and distributed in equal parts to containers. A (first) solution system was then added to the container, wherein this solvent has ten times the mass relative to the fibroin fibers (30 g of fibroin fibers+300 g of first solution system). The first solution system used was a so-called Ajisawa solution which consists of calcium chloride, ethanol and distilled water in a fixed ratio of 1:2:8. By heating the mixture of fibroin fibers and solution system in a water bath at 75° C. and over a period of 60 minutes, the fibroin fibers were dissolved in the first solution system. The containers were shaken vigorously after 15 minutes in each case. In this way, a fibroin raw solution was produced.

For subsequent cleaning, the fibroin fibers were treated with 1 M HCl solution in an amount which corresponded to 1/10 of the mass of the fibroin fibers for 24 hours. The pH of the fibroin raw solution was thereby lowered to a value of <1.

The subsequent neutralization by raising the pH of the fibroin solution to 7.5 was carried out under sterile conditions with the same amount of 1 M NaOH, which was added to the fibroin raw solution.

For desalting, the purified fibroin raw solution was desalted in the sterile dialyzer until the waste water has a permanent conductivity of <500 μS/cm. The solution was concentrated to 10% (mass fraction of the fibroin in the resulting fibroin solution).

The solution resulting therefrom had the following typical properties:

| Appearance | Amber, cloudy solution | |
|---|---|---|
| Molecular weight | 10-400 | kDa |
| pH | 6.5 | |
| Conductivity | 500 | μS/cm |
| Viscosity | 250 | mPa · s |
| Endotoxin value | <1 | EU/ml |
| Bioburden | <5 | CFU |
| DNA content (260:280 nm) | <0.65 | |
| Impurities (e.g., heavy metals) | <50 | ppm |

### Embodiment 4 (Viscose Fibroin Solution)

For degumming, 200 g of silk cocoons of the silkworm were treated in a (second) solution system consisting of 2 l of 0.5 M sodium hydroxide solution for 30 minutes under regular shaking. The container holding the mixture of second solvent and silk cocoons was shaken vigorously after 10 minutes in each case. As a result, the sericin of the silk cocoons was dissolved in the second solution system and thereby separated from the fibrous fibroin. 1 l of 1 M HCl solution was then added for neutralization, and this mixture was reacted for 15 minutes, wherein the vessels were shaken after 5 minutes in each case. The fibroin fibers were then removed from the sericin solution by filtration by means of a sieve.

This was followed by washing and drying the fibroin fibers by swelling them three times for 10 minutes with distilled water and then drying them at 80° C. in an oven for 4 hours (commercially available dehydrator).

The dried fibroin fibers were then weighed and distributed in equal parts to containers. A (first) solution system was then added to the container, wherein this solution system has ten times the mass relative to the fibroin fibers (30 g of fibroin fibers+300 g of first solution system). The first solution system used was a so-called Ajisawa solution which consists of calcium chloride, ethanol and distilled water in a fixed ratio of 1:2:8. By heating the mixture of fibroin fibers and first solution system in a water bath at 75° C. and over a period of 60 minutes, the fibroin fibers were dissolved in the first solution system. The containers were shaken vigorously after 15 minutes in each case. In this way, a fibroin raw solution was produced.

For subsequent purification, the fibroin raw solution was treated for 24 h with 1 M HCl solution in an amount which corresponded to 1/10 of the mass of the dissolved fibroin. The pH of the fibroin raw solution was thereby lowered to a value of <1.

The subsequent neutralization by raising the pH of the fibroin solution to 7.5 was carried out under sterile conditions with the same amount of 1 M NaOH.

For desalting, the purified fibroin raw solution was desalted in a sterile dialyzer until the waste water has a permanent conductivity of <500 μS/cm. The solution was concentrated to 10% (mass fraction of the fibroin in the resulting fibroin solution).

A use of the sericin solution according to the supplementary exemplary embodiment 1 is possible in principle and therefore possible in all exemplary embodiments.

The solution resulting therefrom had the following typical properties:

| Appearance | White, cloudy solution | |
|---|---|---|
| Molecular weight | 10-400 | kDa |
| pH | 8.5 | |
| Conductivity | 400 | μS/cm |
| Viscosity | 50 | mPa · s |
| Endotoxin value | <1 | EU/ml |
| Bioburden | <5 | CFU |
| DNA content (260:280 nm) | <0.65 | |
| Impurities (e.g., heavy metals) | <50 | ppm |

1. A method for generating at least one fibroin solution with the following method steps proceeding in the indicated order:
providing fibroin;
dissolving the fibroin in a first solution system to generate a fibroin raw solution;
cleaning the fibroin raw solution by lowering the pH of the fibroin raw solution by introducing an acidic substance into the fibroin raw solution;
raising the pH of the fibroin raw solution by introducing a basic substance into the fibroin raw solution;
desalting the fibroin raw solution to produce the fibroin solution.

2. The method according to claim 1, characterized in that the dissolving of the fibroin is supported by introducing thermal energy and/or kinetic energy in the first solution system.

3. The method according to claim 1, characterized in that the first solution system comprises an acidic compound, and/or a chaotropic salt compound, and/or a solvent, and/or distilled water.

4. The method according to claim 1, characterized in that the pH of the fibroin raw solution is lowered to less than 2, or less than 1.5, or less than 1.

5. The method according to claim 1, characterized in that the desalting of the fibroin raw solution is accomplished by dialysis, wherein salt molecules diffuse through a semipermeable membrane from the fibroin raw solution into a recipient liquid.

6. The method according to claim 1, characterized in that that the desalting of the fibroin raw solution is carried out until a conductivity value of the fibroin solution of at most 1000 μS/cm, or at most 750 μS/cm, or at most 500 μS/cm, or at most 250 μS/cm is reached.

7. The method according to claim 1, characterized in that the concentration of fibroin in the fibroin solution is set by generating a defined overpressure of the fibroin raw solution during the desalting.

8. The method according to claim 7, characterized in that a concentration of the fibroin of at least 3%, or at least 10%, or at least 20%, or at least 30% is set.

9. The method according to claim 1, characterized in that the provision of the fibroin is accomplished by separating the fibroin and at least one secondary constituent from a silk by introducing the silk into a second solution system which selectively dissolves the secondary constituent, whereby a mixture arises consisting of the fibroin and the solution with the secondary constituent, and by removing the fibroin from the solution with the secondary constituent.

10. The method according to claim 9, characterized in that the second solution system comprises a basic substance.

11. The method according to claim 9, characterized in that the separation of the fibroin and the at least one secondary constituent is assisted by introducing thermal energy and/or kinetic energy.

12. The method according to claim 9, characterized in that that the secondary constituent is sericin.

13. The method according to claim 9, characterized in that that the mixture comprises sodium carbonate, fibroin and distilled water in a ratio that corresponds with a ratio of either 0.06 M, or 0.04 M, or 0.02 M sodium carbonate to 0.2 kg fibroin to 6 l distilled water.

14. The method according to claim 9, characterized in that a mixture consisting of the fibroin and the second solution system is subjected to a temperature between 60° C. and 100° C. over a period of between 30 minutes and 180 minutes.

15. The method according to claim 9, characterized in that the fibroin is washed and/or dried after being removed from the solution with the secondary constituent and before being dissolved in the first solution system.

16. The method according claim 9, characterized in that that one, more or all of the following method steps are performed:
purifying the solution with the secondary component by lowering the pH of the solution with the secondary component by introducing an acidic compound into the solution with the secondary constituent, and/or by heating the solution with the secondary component;
raising the pH of the solution with the secondary component by introducing a basic compound into the solution with the secondary component;
desalting the solution with the secondary constituent.
